(19) United States
(12) Patent Application Publication
(10) Pub. No.: US 2022/0211392 A1
(43) Pub. Date: Jul. 7, 2022

(54) HANDLE FOR MODULAR TOOL

(71) Applicant: OSTIUM GROUP, SAINT ETIENNE DE MONTLUC (FR)

(72) Inventors: Léopold BIBARD, SAINT ETIENNE DE MONTLUC (FR); Edgard SOQUENNE, SAINT ETIENNE DE MONTLUC (FR); Vincent RETAILLEAU, SAINT ETIENNE DE MONTLUC (FR)

(21) Appl. No.: 17/603,544

(22) PCT Filed: Jul. 28, 2020
(86) PCT No.: PCT/EP2020/071301

§ 371 Date: Oct. 13, 2021

(30) Foreign Application Priority Data
Jul. 31, 2019 (EP) 19305991.2

(51) Int. Cl. A61B 17/16; A61B 17/92

(52) U.S. Cl. CPC A61B 17/1659; A61B 2017/00464; A61B 17/92

(57) ABSTRACT

A handle for a modular tool is provided in which the handle may be releasably coupled to a working part such as a rasp, reamer or impactor, or to a secondary element which may itself be connected to a working part. The coupling comprises a cylindrical member provided with one or more external helical thread, and a lateral slot opening on one periphery of the handle. A threaded ring has threads engaging the threads of the cylindrical member, and is rotatable about the cylindrical member between an extended position in which said ring obstructs the slot opening, and a retracted position in which the ring leaves the slot opening unobstructed.

### FIELD OF THE INVENTION

The present invention relates to modular tools, and in particular a handle element for such tools.

### BACKGROUND OF THE INVENTION

The concept of modularity in tools is almost as old as tool use itself. Many ancient tool designs such as hammers, axes, picks and the like comprise a working element fixed to a handle by some means which allows the periodic replacement of one element or the other. Developments in manufacturing processes over the last two hundred years meanwhile have made it possible to develop releasable fixing mechanisms. For example, screw driver heads are commonly available as 6.35 mm (quarter inch) hexagon bits, which may be inserted as required in a standard handle. The bits may be secured magnetically, or by means of a spring loaded ball bearing engaging a groove in the bit. In some contexts meanwhile, the operational constraints extant in that context may tend to lead away from such approaches. In the field of surgical instruments for example, the high forces involved along with sterilisation requirements may tend to indicate an all-in-one approach.

FIG. 1 shows an orthopaedic reamer as known in the state of the art.

As shown in FIG. 1, a reamer 100 comprises a handle 102 with a strike head 101 and a reamer working surface 103. The reamer 100 is typically made entirely of stainless steel.

Notwithstanding the foregoing, the cost of manufacturing a complete set of stand-alone tools for example as shown in FIG. 1 can be considerable, and even in the field of surgical tools, some attempts at modular tools are known.

In this, as in any field where a solid positive engagement between the handle and further elements is a critical requirement, special consideration must be given to the securing mechanism.

FIG. 2 shows a releasable securing mechanism for modular tools as known in the state of the art.

FIG. 2 shows a conventional securing mechanism known in the art as a “Hudson Fitting”. In particular, FIG. 2 shows a male Hudson fitting 200, attached to a tool element 201. The fitting comprises a cylindrical member 203 with a semi-circular channel or groove 204. When the cylindrical member 203 is slid into the corresponding female element, a spring loaded ball bearing 205 engages the channel so as to prevent accidental decoupling. In implementations where uncoupling must be avoided in the presence of a separating force, the ball bearing may be replaced with a removable cotter pin or the like. As shown, the fitting also comprises a flattened flange 202 at the proximal end of the fitting closest to the tool element 201. The flats of this flange may engage corresponding surfaces on the corresponding female element when the coupling is fully inserted, so that rotational forces may be effectively transferred between the two elements of the coupling.

Fittings such as that shown in FIG. 1 have been found unsatisfactory in terms of their ability to provide a solid positive engagement between the handle and further elements, whilst supporting uncoupling with a minimum of force and dexterity when required, yet averting the risk of accidental uncoupling. As such, it is desired to provide an improved coupling addressing some or all of these concerns.

### SUMMARY OF THE INVENTION

In accordance with the present invention in a first aspect there is provided a handle for a modular tool comprising a body having a distal end and a proximal end. The distal end provides a releasable coupling for an element of the modular tool, the releasable coupling comprising a cylindrical member provided with a first external helical thread, and a lateral slot. The slot opens on one periphery of the distal end of the body and widens from the distal end towards the proximal end of the handle. The releasable coupling further comprises a threaded ring, the threads of the ring engaging the first external helical thread of the cylindrical member, the threaded ring being rotatable about the distal end of the member between an extended position in which the ring obstructs the slot opening on one periphery of the distal end of the body, and a retracted position in which the ring leaves the slot opening on one periphery of the distal end of the body unobstructed.

In accordance with a development of the first aspect, the lateral slot defines a T slot or dovetail.

In accordance with a further development of the first aspect, the dimensions of the lateral slot vary radially so that the force required to slide a corresponding keyed element of the secondary element into the lateral slot increases as the corresponding keyed element of the tool progresses into the slot.

In accordance with a further development of the first aspect, the angular displacement of the threaded ring when rotated between the extended position and the retracted position is between 170 and 190 degrees.

In accordance with a further development of the first aspect, the threaded ring comprises an indentation at the distal edge thereof, the indentation being positioned on the circumference of the ring such that when the ring is in the retracted position the indentation is aligned with the slot.

In accordance with a further development of the first aspect, the cylindrical member is provided with a second external helical thread parallel the first external helical thread, and wherein the threads of the ring engage both the first external helical thread and the second external helical thread of the cylindrical member.

In accordance with a further development of the first aspect, the first helical thread has a first root to crest ratio, and the second helical thread has a second root to crest ratio, wherein the threads of the ring are defined so that the ring can only engage the corresponding respective helical thread, and thereby may only be mounted on the handle in one configuration.

In accordance with a further development of the first aspect, the pitch, the minor diameter, the major diameter, or the root to crest ratio of the of the first external helical thread varies along the length of the first external helical thread so that the force required to rotate the ring increases as the ring is rotated about the distal end of the member from the retracted position to the extended position.

In accordance with a further development of the first aspect, modular tool comprises a surgical instrument.

In accordance with a further development of the first aspect, modular tool is for surgery of the orthopaedic surgery or bone traumatology.

In accordance with a further development of the first aspect, modular tool comprises a rasp or reamer or impactor.

In accordance with a further development of the first aspect, the modular tool comprises the handle, an interface part being releasably coupled to the handle and a working part being releasably coupled to the interface part.

In accordance with a further development of the first aspect, the handle is composed of a synthetic material or a synthetic composite material.

In accordance with a further development of the first aspect, the handle is composed of a glass fibre reinforced polyarylamide.

### BRIEF DESCRIPTION OF THE DRAWINGS

The above and other advantages of the present invention will now be described with reference to the accompanying drawings, for illustration purposes only, in which:

FIG. 1 shows an orthopaedic reamer as known in the state of the art;

FIG. 2 shows a releasable securing mechanism for modular tools as known in the state of the art;

FIG. 3 shows a handle for a modular tool in accordance with an embodiment;

FIG. 4a shows the handle in a first, retracted position prior to the insertion of the secondary element 305;

FIG. 4b shows the handle in a first, retracted position after insertion of the secondary element 305;

FIG. 4c shows the handle in a second, extended position after insertion of the secondary element 305;

FIG. 5a shows a sectional view of the slot in a first embodiment;

FIG. 5b shows a sectional view of the slot in a second embodiment;

FIG. 6 shows a plan view of a keyed element corresponding to the slot of FIG. 5a;

FIG. 7 shows a tool in accordance with an embodiment;

FIG. 8a shows further detail of the embodiment of FIGS. 3 to 7;

FIG. 8b shows a variant of the arrangement of FIG. 8a.

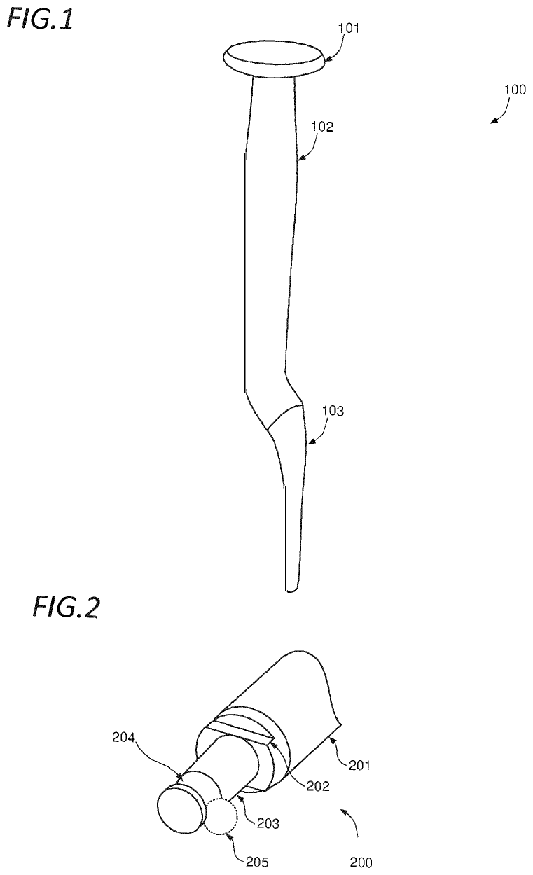

### DETAILED DESCRIPTION

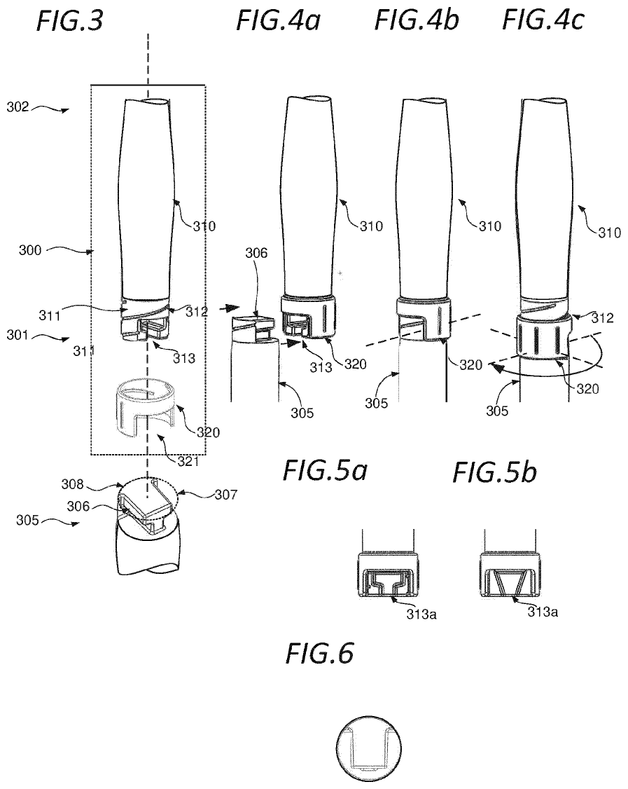

FIG. 3 shows a handle for a modular tool in accordance with an embodiment. As shown, the handle 300 is provided with a body 310 having a distal end 301 and a proximal end 302. The distal end 301 provides a releasable coupling for a secondary element 305 of the modular tool. The releasable coupling comprises a cylindrical member 311 provided with a first external helical thread 312, and a lateral slot 313, the slot opening on one periphery of the distal end 301 of the body 310, the slot widening from the distal end 301 towards the proximal end 302. The releasable coupling further comprises an internally threaded ring 320, the threads of the ring 320 engaging the first external helical thread 312 of the cylindrical member 311, the threaded ring 320 being rotatable about the distal end 301 of the cylindrical member 311.

While the handle is shown substantially as a cylinder with its major axis extending from the distal to proximal end, the skilled person will appreciate that other forms may be provided depending on the intended use of the handle. In particular, the handle may alternatively be “T” shaped, with the cross bar of the “T” at the proximal end of the handle so that the user may grip the handle for example with the index and middle finger on one side of the vertical, and the ring and small finger on the other side. Similarly, the handle may alternatively be “D” shaped, with the vertical of the “D” at the proximal end of the handle so that the user may grip the handle at right angles (or some other angle as required) to the major axis of the handle.

Where a plurality of helical threads are provided, one or more of these threads may have dimensions different to other threads, such that the ring can only engage the cylindrical member in the desired orientation, for example so as to ensure proper positioning of the optional detent with respect to the slot in the extended and retracted positions. For example, the helical thread has a first root to crest ratio, and the second helical thread has a second root to crest ratio, wherein the threads of the ring are defined so that the ring can only engage the corresponding respective helical thread, and thereby may only be mounted on the handle in one configuration.

Similarly, the circumferential spacing of the threads may be varied to achieve the same effect.

The threads on the cylindrical member and/or the ring need not be continuous.

As shown, the ring is provided with an optional detent 321, in the distal circumferential edge thereof.

It will be appreciated that there is provided a secondary element having features in correspondence to the handle of the present invention, and in particular to the releasable coupling thereof. In particular, there is provided a secondary element for a modular tool, the secondary element having a distal end and a proximal end disposed on a longitudinal axis, the distal end of said secondary element being enclosed within a notional cylinder 307. The secondary element comprises a first keyed element 306 widening in a distal direction. A trailing edge 308 of the keyed element conforms to the cylinder 307, whereby said secondary element may be secured in a corresponding handle by inserting said first keyed element 306 with the trailing edge 308 outward into a corresponding slot defined in said handle, whereby a ring 320 of said handle corresponding to said cylinder may be slid into position so as to block said keyed element 306 in said handle.

The trailing edge 308 of the keyed element which conforms to the cylinder may furthermore be partially threaded so as to provide a continuous thread with threads provided in a cylindrical member of the handle, and adapted to engage threads of the ring as described above.

FIGS. 4a, 4b and 4c show the handle of FIG. 3 in different configurations.

As shown, the threaded ring 320 is rotatable about the distal end 301 of the member 311 from a first, retracted position as shown in FIGS. 4a and 4b, to a second, extended as shown in FIG. 4c.

FIG. 4a shows the handle in a first, retracted position prior to the insertion of the secondary element 305. The secondary element may comprise a tool. For example, the tool may comprise a surgical instrument. More particularly for example, the tool may be for surgery of the orthopaedic surgery or bone traumatology. Still more particularly for example tool may comprise a rasp or reamer or impactor. Alternatively, the secondary element may comprise an intermediate adaptor between the handle and a further component, where the further component may comprise a tool as discussed above or otherwise.

As shown, the secondary element 305 comprises a tongue or keyed element 306 which has a form complementary to the slot 313. As shown the ring 320 is in a retracted position in which the ring leaves the slot opening on one periphery of the distal end of the body unobstructed. By this means, the keyed element 306 of the secondary element 306 may be introduced into the slot 313 of the handle.

As shown, the optional detent 321 aligns with the slot in the retracted position.

FIG. 4b shows the handle in a first, retracted position after insertion of the secondary element 305.

Since the slot 313 widens from the distal end 301 towards the proximal end 302, a correspondingly formed keyed element 306 may be slid into the slot laterally as shown in FIG. 4b, but once in position, will not be movable along the axis of the handle, i.e. towards or away from the distal end of the handle, but only back or forth along the axis of the slot.

FIG. 4c shows the handle in a second, extended position after insertion of the secondary element 305. As shown in FIG. 4c, the ring has been rotated about the cylindrical member 311, so that through the engagement of the threads of the ring in the threads 312 of the cylindrical member, this rotational movement has been translated into a linear movement towards the distal end of the handle, so that the ring obstructs the slot opening on one periphery of the distal end of the body.

As shown, this movement comprises a rotation of 180° clockwise about the axis of the handle when viewed down the length from the proximal to the distal end of the handle. The skilled person will appreciate that the pitch of the threads and the angle through which the ring is rotated will determine the distance along the cylindrical member that the ring 320 travels. On one hand it is desirable that the distance travelled should be as great as possible, to allow the use of a long keyed element 306, providing a strong and rigid connection between the handle and secondary element. On the other hand, in use it will be desirable that the user be required to turn the circle through as small an angle as possible, so that the manipulation may be performed with the lowest possible demand on the user's dexterity. The chosen compromise between these considerations is defined by the pitch of the respective threads of the ring and the cylindrical member. On this basis, the angular displacement of the ring between the extended position and the retracted position is preferably less than 361°, more preferably less than 271°, more preferably less than 181°. A multiple of 90° may be advantageous in terms of being more intuitive to the user. The angular displacement of the ring between the extended position and the retracted position is preferably more than 89°. The chosen compromise between these considerations is defined by the pitch of the respective threads of the ring and the cylindrical member.

Additional constraints may occur through a desire to limit the likelihood of the ring moving along the cylindrical member under its own weight, or in response to an accidental or incidental pressure, which will tend to favour a tighter pitch, although these considerations may also be managed by selecting materials, surfaces and/or tolerances so that the friction between elements reduces the risk of such unwanted movement. On this basis, the threads of the ring and cylindrical member may be configured in accordance with an embodiment such that the ring is turned through substantially one single revolution or less between the extended position and the retracted position. In accordance with a further embodiment the ring is turned through substantially one half revolution or less between the extended position and the retracted position. In accordance with a further embodiment the ring is turned through substantially one quarter revolution or less between the extended position and the retracted position. In accordance with a further embodiment the ring is turned through substantially one third revolution or more between the extended position and the retracted position. Meanwhile, the threads of the ring and cylindrical member may be configured in accordance with an embodiment such that the ring moves along the axis of the cylindrical member a distance substantially equal to 2 cm or less between the extended position and the retracted position. In accordance with a further embodiment the ring moves along the axis of the cylindrical member a distance substantially equal to 1.5 cm or less between the extended position and the retracted position. In accordance with a further embodiment the ring moves along the axis of the cylindrical member a distance substantially equal to 1 cm or less between the extended position and the retracted position. The skilled person will appreciate that any combination of angle of rotation and linear displacement may be selected within these ranges, and indeed outside these ranges. It may be noted that the thread of the cylindrical member and ring of FIGS. 4a, 4b and 4c comprises a double thread. A double thread may be advantageous in providing a firm engagement between the threads of the ring and the cylinder while permitting a short threaded section along the length of the ring, so as to minimise the dimensions of the ring. The skilled person will appreciate that a single thread may also be adequate in many implementations. The skilled person will appreciate that a three or even more threads may be appropriate in other implementations.

As shown in FIGS. 3 and 4a, the slot 313 defines substantially a T cross section, and the keyed element 306 is shaped correspondingly. This shape achieves the objective of ensuring that the keyed element can only enter or exit the slot through the lateral opening of the cylindrical section, and that once the ring is in the extended position, no force in any direction on the secondary element with respect to the handle will separate the secondary element from the handle. The skilled person will recognise that the keyed element and corresponding slot may have any form complying with the general requirement that it widens from the distal end towards the proximal end. As such, it may form a wedge, dovetail or T section as described above. It may furthermore be circular, elliptical, rectangular, square, or any other form. The keyed element will generally constitute an extrusion of the chosen cross section from one side to the other. In certain embodiments, the keyed element may taper from one side to the other. Where this is the case the slot may taper from side to side correspondingly. Where this is the case, the slot may be defined as being deeper from side to side that the length from side to side of the corresponding keyed element. On this basis, the tapering walls of the keyed element will engage the sides of the keyed element before the end of the keyed element reached the lateral extremity of the slot. By this means, the slot will become progressively tighter as the keyed element is inserted, and a firm insertion without any play between the handle and secondary element may be achieved by pushing the keyed element fully into the slot.

FIG. 4c shows the handle in a second, extended position after insertion of the secondary element 305.

As shown, the ring 320 has been rotated through 180° clockwise about the cylindrical member 311, and by the action of the engagement between the threads 312 on the cylindrical member 311 with those of the ring, the ring has progressed along the length of the cylindrical member so as to block the slot 313. It may further be noted that while in the retracted position as shown in FIGS. 4a and 4b the optional detent 321 aligns with the slot, in the extended position of FIG. 4c the optional detent 321 no longer aligns with the slot. As such, the provision of the slot 321 means that the slot can be effectively blocked by a smaller linear movement of the ring.

As such, as shown the keyed element of the secondary element 305 is entirely trapped in the slot by the new position of the ring, as the result of a simple half turn (in the present example) of the ring, which may be effectuated by the user with a movement of the thumb, retaining the tool in the other fingers of the actuating hand, and leaving the other hand free. To further facilitate this action, the ring may be knurled, grooved, provided with a non-slip coating, provided with flats or otherwise treated to improve the users grip thereon. Where the ring is provided with flats, these may be even in number, and may further be dimensioned so that they may be engaged using a spanner of standard dimensions, for example as defined in ISO/TC 29/SC 10 and the like.

In certain embodiments, the shape or dimension of ring and cylindrical member, and in particular the thread on either or both of the ring or the cylindrical member may vary along their length, for example such that their engagement becomes progressively tighter towards the extended position, so that while it may move freely at certain portions of its travel, as it approaches the extended position it becomes somewhat resistant to movement. This approach may be advantageous in further reducing the risk of the ring being moved from the extended position inadvertently, which might otherwise lead to a loosening or even decoupling of the secondary element from the handle. Similarly, the engagement between the ring and cylindrical member may become progressively tighter towards the retracted position, so that while it may move freely at certain portions of its travel, as it approaches the retracted position it becomes somewhat resistant to movement. This approach may be advantageous in further reducing the risk of the ring being moved from the retracted position inadvertently, which might otherwise complicate the task of inserting the keyed element of the secondary element. The variations in shape or dimensions may include for example variations in the pitch, the minor diameter, the major diameter, or the root to crest ratio or any combination of these. Additionally or alternatively a similar effect may be achieved by a variation in the outer diameter of the cylindrical member and/or the inner diameter of the ring along the length thereof, for example so that as the ring moves towards the extended position, it becomes progressively tighter on the cylindrical member and/or so that as the ring moves towards the retracted position, it becomes progressively tighter on the cylindrical member.

As discussed above, the slot (and correspondingly the keyed element) widens from the distal end towards the proximal end. As discussed above, the slot, and correspondingly the keyed element may take many forms whilst satisfying this requirement. FIGS. 5a and 5b show two possible such implementations.

FIG. 5a shows a sectional view of the slot in a first embodiment.

As shown in FIG. 5a, the slot 313a is generally T shaped in cross section. It may be noted that the shoulders defining the top of the narrow stem of the T shape are not entirely parallel with the flat top of the T shape. In other embodiments, these shoulders may be parallel with the top, or adopt any other angle as may be found to be expedient.

FIG. 5b shows a sectional view of the slot in a second embodiment.

As shown in FIG. 5b, the slot 313b is generally in the form of a dovetail in cross section.

As shown in both FIGS. 5a and 5b, the dimensions of the lateral slot vary radially (that is, from the edge towards the middle) so that the force required to slide a corresponding keyed element of the tool into the lateral slot increases as the corresponding keyed element of the tool progresses into the slot.

FIG. 6 shows a plan view of a keyed element corresponding to the slot of FIG. 5a.

The keyed element of FIG. 6 corresponds to that of the secondary element 305 shown in FIG. 3. As shown, the slight tapering of the keyed element is visible.

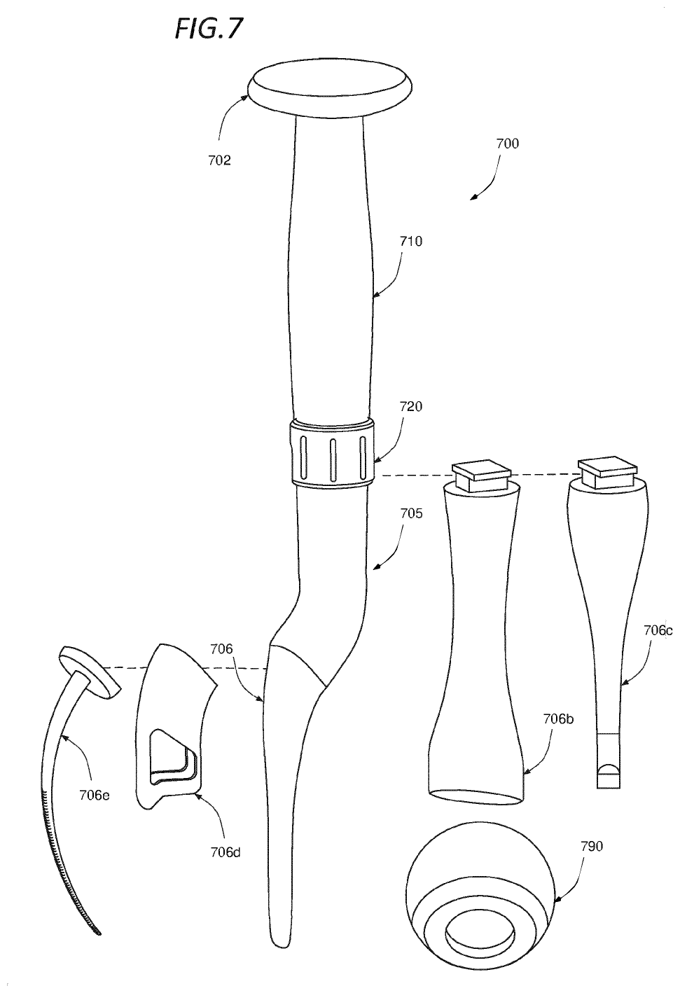

FIG. 7 shows a tool in accordance with an embodiment.

As shown, there is provided a modular tool 700 comprising a handle 710 substantially as described above, and a secondary element 705 substantially as described above. The handle 710 comprises a releasable coupling substantially as described above, and in particular comprising a threaded ring 720, the threads of the ring engaging an external helical thread of the cylindrical member of the handle 700. The threaded ring 720 is rotatable about the distal end of the member between an extended position as shown in which the ring obstructs a slot opening on one periphery of the distal end of the body, and a retracted position in which the ring leaves the slot opening on one periphery of the distal end of the body unobstructed.

The handle may optionally be provided with an angle datum such as a radial line on the guard plate, or a radial lumen through which a bar may by inserted.

As shown, the handle further comprises an optional guard plate 702 at the proximal end thereof. Such a guard plate may serve to protect the hand of a user when gripping the handle 710 from blows struck against the proximal end thereof with a hammer, mallet or the like, for example where the tool 705 is a chisel, reamer or other such tool requiring a percussive application.

One field in which a handle as described may be appropriate is that of surgical instruments, such that the modular tool as a whole may comprise or constitutes a surgical instrument. More particularly, the modular tool may be for orthopaedic surgery or bone traumatology. More particularly, the modular tool may be is for surgery of the hip, shoulder or knee. More particularly, as shown, a working part 706, and thus the modular tool as a whole 700 comprises a rasp or reamer or impactor. It will be appreciated that in line with the many fields of application and associated tool types that may be envisaged, many different possible working parts 706 may be envisaged, for use with a single handle in accordance with embodiments as described above. Further examples of possible working parts, and resulting modular tools, include head impactor 706b (adapted to exert a force on an implant head 790), implant impactor 706c, which are examples of working parts that may be attached directly to the handle without a separate intervening secondary element 705, a curved rasp 706e, osteotome 706d which are further examples of working parts that may be attached directly to the handle by means of a separate intervening secondary element 705 and many other tools as will readily occur to the skilled person.

While the working part may incorporate the keyed part as described above, there may alternatively be provided a separate interface part as described above being releasably coupled to the handle as described above, and a working part being releasably coupled to the interface part, by a further releasable coupling means, which may comprise an additional ring mechanism as described above, a Hudson fitting as known in the state of the art, or any other convenient mechanism.

The handle of the present invention may be formed of any material. In particular, it may be formed of steel, aluminium, titanium or any other suitable metal or alloy. It may also be formed of a thermoplastic or other synthetic material. It may in particular be formed from a polyamide, for example a polyarylamide. The synthetic material may comprise additional components such as a filler, swelling agent and the like. It may still further be formed of a synthetic composite material, comprising a glass, carbon fibre, carbon nanoparticle or any other material exhibiting a high tensile strength, in a matrix of a synthetic material, such as any of those listed above. In certain embodiments, the handle of claim 13 wherein the handle is composed of a glass fibre reinforced polyacrylamide, such as for example that marketed by the Solvay corporation under the trademark “Ixef 1022” or more particularly “Ixef GS 1022”.

The handle may be formed of different materials in different regions, including metal parts and synthetic parts. The handle may also comprise voids for the purpose of economy of material, reduced weight and so on.

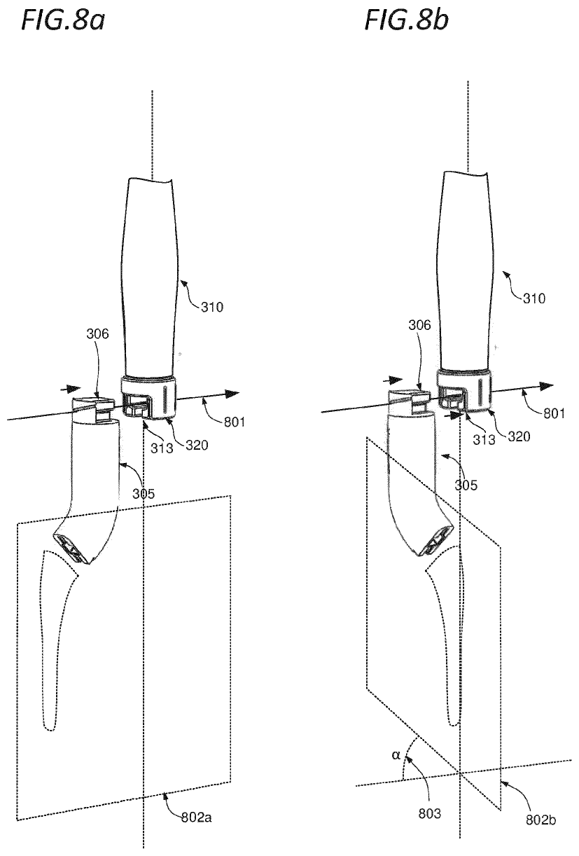

FIG. 8a shows further detail of the embodiment of FIGS. 3 to 7.

In particular, FIG. 8a shows elements 305, 306, 310, 313 and 320 as described above with reference to FIG. 3. Meanwhile, as shown a line 801 represents the axis along which a secondary element 305 as described herein may be slid into the coupling element 313. Meanwhile, element 802a defines a plane aligned with the working plane in with which the longitudinal axis of the handle secondary element, and/or tool element mounted therein is aligned. As shown in FIG. 8a, the plane 802a is also aligned with the line 801.

FIG. 8b shows a variant of the arrangement of FIG. 8a.

In particular, FIG. 8b shows elements 305, 306, 310, 313 and 320 as described above with reference to FIG. 3. Meanwhile, as shown a line 801 represents the axis along which a secondary element 305 as described herein may be slid into the coupling element 313. Meanwhile, element 802a defines a plane aligned with the working plane in with which the longitudinal axis of the handle secondary element, and/or tool element mounted therein is aligned. As shown in FIG. 8b, the plane 802a is at an angle α, designated by element 803, with respect to the line 801, as rotated about the axis of the handle, so that the secondary element 305 as described herein may be seen as being slid sideways in into into the coupling element 313 of the handle.

This approach has the advantage with respect to that of FIG. 8a that forces exerter in the working plane 802b will not tend to disassociate the secondary element from the handle as may be the case with the configuration of FIG. 8a.

As shown in FIG. 8b the angle α the angle is 90°, so that the secondary element is typically inserted from the user's right. This may be convenient for a left handed user holding the handle in their left hand, and using their right hand to insert the tool holder. The skilled person may select an angle α as may be convenient. For example the angle α the angle may be selected as 270°, so that the secondary element is typically inserted from the user's left. This may be convenient for a right handed user holding the handle in their left hand, and using their right hand to insert the tool holder.

Angles smaller than 90° or greater than as 270° may mean that while forces in the working plane have a component in the insertion axis 801, this will tend to push the secondary element further into the coupling.

Angles between 90° and 270° may mean that a user can conveniently insert the secondary element by bringing the secondary element towards themselves, which may be easier in a constrained space.

The skilled person will recognise that intermediate angles may be selected as appropriate in view of the particular intended use conditions, which may combines these and other benefits in desired proportions.

Accordingly, there is provided a handle for a modular tool as described above wherein the modular tool has a working plane, and wherein the axis of said lateral slot is at an angle with respect to said working plane.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed. The subject matter of the present disclosure includes all novel and non-obvious combinations and sub-combinations of the various processes, systems and configurations, and other features, functions, acts, and/or properties disclosed herein, as well as any and all equivalents thereof.

Accordingly there is provided a handle for a modular tool in which the handle may be releasably coupled to a working part such as a rasp, reamer or impactor, or to a secondary element which may itself be connected to a working part. The coupling comprises a cylindrical member provided with one or more external helical thread, and a lateral slot opening on one periphery of the handle. A threaded ring has threads engaging the threads of the cylindrical member, and is rotatable about the cylindrical member between an extended position in which the ring obstructs the slot opening, and a retracted position in which the ring leaves the slot opening unobstructed. Accordingly, when the ring is in the retracted position a keyed element of the working part or secondary element may be slide from the side into the slot of the handle, and then once the ring is rotated to the extended position, the working part or secondary element is locked in place.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A handle for a modular tool, the handle comprising
a body having a distal end and a proximal end, said distal end providing a releasable coupling for an element of said modular tool, said releasable coupling comprising:
a cylindrical member provided with
a first external helical thread, and
a lateral slot, said slot opening on one periphery of said distal end of said body, said slot widening from said distal end towards said proximal end,
said releasable coupling further comprising a threaded ring, the threads of said ring engaging said first external helical thread of said cylindrical member, said threaded ring being rotatable about said distal end of said member between an extended position in which said ring obstructs said slot opening on one periphery of said distal end of said body, and a retracted position in which said ring leaves said slot opening on one periphery of said distal end of said body unobstructed.

2. The handle of claim 1 wherein said lateral slot defines a T slot or dovetail.

3. The handle of claim 1 wherein the dimensions of said lateral slot vary radially so that the force required to slide a corresponding keyed element of said secondary element into said lateral slot increases as said corresponding keyed element of said tool progresses into said slot.

4. The handle of claim 1 wherein the angular displacement of said threaded ring when rotated between said extended position and said retracted position is between 170 and 190 degrees.

5. The handle of claim 1 wherein said threaded ring comprises an indentation at the distal edge thereof, said indentation being positioned on the circumference of said ring such that when said ring is in said retracted position said indentation is aligned with said slot.

6. The handle of claim 5 wherein said cylindrical member is provided with a second external helical thread parallel said first external helical thread, and wherein the threads of said ring engage both said first external helical thread and said second external helical thread of said cylindrical member.

7. The handle of claim 6 wherein said first helical thread has a first root to crest ratio, and said second helical thread has a second root to crest ratio, wherein the threads of said ring are defined so that said ring can only engage the corresponding respective helical thread, and thereby may only be mounted on said handle in one configuration.

8. The handle of claim 1 wherein the pitch, the minor diameter, the major diameter, or the root to crest ratio of said of said first external helical thread varies along the length of said first external helical thread so that the force required to rotate said ring increases as the ring is rotated about said distal end of said member from said retracted position to said extended position.

9. The handle of claim 1 wherein said modular tool comprises a surgical instrument.

10. The handle of claim 9 wherein said modular tool is for orthopaedic surgery or bone traumatology.

11. The handle of claim 1 wherein said modular tool comprises a rasp or reamer or impactor

12. The handle of claim 1 wherein said modular tool comprises said handle, a secondary element being releasably coupled to said handle and a working part being releasably coupled to said secondary element.

13. The handle of claim 1 wherein said handle is composed of a synthetic material or a synthetic composite material.

14. The handle of claim 13 wherein said handle is composed of a glass fiber reinforced polyarylamide.

15. A secondary element for a modular tool, said secondary element having a distal end and a proximal end disposed on a longitudinal axis, the distal end of said secondary element being enclosed within a cylinder, said secondary element comprising a first keyed element widening in a distal direction. Where a trailing edge of said keyed element conforms to said cylinder, whereby said working part may be secured in a corresponding handle by inserting said first keyed element with said trailing edge outward into a corresponding slot defined in said handle, whereby a ring of said handle corresponding to said cylinder may be slid into position so as to block said keyed element in said handle.
